(19) United States
(12) Patent Application Publication
(10) Pub. No.: US 2016/0312171 A1
(43) Pub. Date: Oct. 27, 2016

(54) A CELL CULTURING PLATFORM, A CELL CULTURE SYSTEM, AND A METHOD FOR MODELING MYELINATION IN VITRO

(71) Applicants: TAMPEREEN YLIOPISTO, Tampereen Yliopisto (FI); TTY-SÄÄTIÖ, Tampere (FI)

(72) Inventors: Susanna NARKILAHTI, Kangasala (FI); Laura YLA-OUTINEN, Tampere (FI); Lassi SUKKI, Tampere (FI); Tomi RYYNANEN, Tampere (FI); Meeri MAKINEN, Tampere (FI); Anu HYYSALO, Tampere (FI); Joose KREUTZER, Tampere (FI)

(21) Appl. No.: 15/104,682

(22) PCT Filed: Dec. 17, 2014
(86) PCT No.: PCT/FI2014/051017

§ 371 Date: Jun. 15, 2016

(30) Foreign Application Priority Data
Dec. 20, 2013 (FI) 20136312

(51) Int. Cl. C12M 1/34; C12M 3/00; C12N 5/0793; C12N 5/079; C12M 3/06; C12M 1/00

(52) U.S. Cl. CPC C12M 41/46; C12M 23/16; C12M 23/22; C12M 23/34; C12N 5/0619; C12N 5/0622; C12M 21/08

(57) ABSTRACT

A cell culturing platform for modeling myelination in vitro comprises first cell chambers (102a, 102b) for containing somas of neuronal cells, a process chamber (103) connected to the first cell chambers with first channels, and a second cell chamber (105) for containing somas of the myelinating cells and connected with second channels to the process chamber. The first channels allow processes of the neuronal cells to enter the process chamber and inhibit the somas of the neuronal cells from moving from the first cell chambers to the process chamber. The second channels allow processes of the myelinating cells to enter the process chamber and inhibit the somas of the myelinating cells from moving from the second cell chamber to the process chamber. Hence, the process chamber, where the myelination takes place, is substantially free from the cell somas and thus detection of the myelination is facilitated.

### FIELD OF THE INVENTION

The invention relates generally to modeling myelination in vitro. More particularly, the invention relates to a cell culturing platform, to a cell culture system, and to a method for modeling the myelination in vitro.

### BACKGROUND

Myelin is material that forms layers, i.e. myelin sheaths, around neuronal cell processes, i.e. around axons of neuronal cells. The myelin is essential for the proper functioning of the nervous system. The production of the myelin sheath is called myelination. In humans, the production of myelin begins in the 14th week of foetal development, and thus myelin exists in the brain already at the time of birth. During infancy, myelination occurs quickly and continues through the adolescent stages of life. Myelinated axons are white in appearance, hence the “white matter” of the brains. The fat helps to insulate the axons from electrically charged atoms and molecules. These charged particles are found in the fluid surrounding the entire nervous system. Myelin is also a part of the maturation process leading to child's fast development, including crawling and walking in the first year. Demyelination is the loss of the myelin sheath insulating the nerves, and it is an indicator of some neurodegenerative autoimmune diseases, including multiple sclerosis “MS”, acute disseminated encephalomyelitis, Neuromyelitis Optica, transverse myelitis, chronic inflammatory demyelinating polyneuropathy, Guillain-Barré syndrome, central pontine myelinosis, inherited demyelinating diseases such as leukodystrophy, and Charcot-Marie-Tooth disease. Demyelination can also occur after traumatic injury such as spinal cord injury as a secondary degeneration process.

Therapeutic interventions to prevent demyelination, i.e. myelin loss, include neuroprotection based on drugs, enhancement of endogenous myelin formation based on drugs, and replacement of lost cells, i.e. transplantation.

In vitro models of myelination are useful in testing and development of drugs and in development of transplantation therapies. Publication Park J., Koito H., Li J., Han A.: High-throughput compartmentalized CNS neuron culture platform for axon degeneration/regeneration study, 14th International Conference on Miniaturized Systems for Chemistry and Life Sciences 3-7 Oct. 2010, Groningen, The Netherlands, pp. 860-262 presents a cell culturing platform suitable for modelling the myelination in vitro. The cell culturing platform is a multi-compartment microfluidic co-culture platform composed of one soma compartment for neurons and six axon compartments. The soma compartment and axon compartments are connected by arrays of axon-guiding channels that function as physical barriers to confine neuronal somas in the soma compartment, while allowing axons to grow into the axon compartments. The oligodendrocytes are loaded into the axon compartments and they can interact with axons but not with the neuronal somas. In some cases it can be, however, challenging to detect whether or not the myelination has taken place in the axon compartments.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying and non-limiting embodiments of the invention.

In accordance with the invention, there is provided a new cell culturing platform suitable for culturing e.g. neuronal cells and myelinating cells so as to model myelination in vitro. The term “myelination” includes at least the following processes: 1) myelination, 2) demyelination and 3) remyelination which can all be modeled in this new culturing platform. A cell culturing platform according to the invention comprises solid material adapted to constitute:
one or more first cell chambers for containing somas of neuronal cells,
a process chamber connected to the one or more first cell chambers with one or more first channels, and
a second cell chamber for containing somas of myelinating cells and connected with one or more second channels to the process chamber.

The one or more first channels are suitable for guiding growth of neuronal cell processes of the neuronal cells from the one or more first cell chambers to the process chamber and for inhibiting the somas of the neuronal cells from moving from the one or more first cell chambers to the process chamber. The process chamber constitutes a room for the myelination of the neuronal cell processes by myelinating cell processes of the myelinating cells. The above-mentioned second channels are suitable for guiding growth of the myelinating cell processes of the myelinating cells from the second cell chamber to the process chamber and inhibiting the somas of the myelinating cells from moving from the second cell chamber to the process chamber. Hence, the process chamber, where the myelination takes place, is substantially free from the cell somas and thus the detection of the myelination is facilitated. The process chamber has an elongated shape so that each of the one or more first channels is connected to one of shorter sides of process chamber and each of the one or more second channels is connected to one of longer sides of the process chamber, the longer sides of the process channel being at least three times longer than the shorter sides of the process channel.

In accordance with the invention, there is provided also a new cell culture system for modeling myelination in vitro. A cell culture system according to the invention comprises a cell culturing platform according to the invention, wherein:
each of the one or more first cell chambers contains somas of neuronal cells,
neuronal cell processes of the neuronal cells are capable of entering the process chamber through the one or more first channels and forming contacts including synapses with each other,
the second cell chamber contains somas of myelinating cells, and
myelinating cell processes of the myelinating cells are capable of entering the process chamber through the one or more second channels and of myelinating the neuronal cell processes of the neuronal cells in the process chamber.

In accordance with the invention, there is provided also a new method for modeling myelination in vitro. A method according to the invention comprises culturing neuronal cells and myelinating cells in a cell culturing platform according to the invention, wherein:
each of the one or more first cell chambers contains somas of the neuronal cells,
neuronal cell processes of the neuronal cells enter the process chamber through the one or more first channels and form synapses with each other,
the second cell chamber contains somas of the myelinating cells, and
myelinating cell processes of the myelinating cells enter the process chamber through the one or more second channels and myelinate the neuronal cell processes of the neuronal cells in the process chamber.

A number of exemplifying and non-limiting embodiments of the invention are described in accompanied dependent claims.

Various exemplifying and non-limiting embodiments of the invention both as to constructions and to methods of operation, together with additional objects and advantages thereof, will be best understood from the following description of specific exemplifying embodiments when read in connection with the accompanying drawings.

The verbs “to comprise” and “to include” are used in this document as open limitations that neither exclude nor require the existence of also un-recited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

The exemplifying and non-limiting embodiments of the invention and their advantages are explained in greater detail below with reference to the accompanying drawings, in which:

FIG. 1a shows a top-view of a cell culturing platform according to an exemplifying and non-limiting embodiment of the invention,

FIG. 1b shows a partial magnification of the cell culturing platform,

FIG. 1c shows a top view of the cell culturing platform when provided with a cover portion,

FIG. 1d shows a view of a section taken along the line A-A shown in FIG. 1c,

FIG. 2 illustrates a cell culture system according to an exemplifying and non-limiting embodiment of the invention for modeling myelination in vitro,

FIG. 3 shows a chart of a method according to an exemplifying and non-limiting embodiment of the invention for modeling myelination in vitro,

FIGS. 4a and 4b show images which illustrate a first example case,

FIGS. 5a, 5b, and 5c show images which illustrate a second example case, and

FIGS. 6a, 6b, and 6c show images which illustrate a third example case.

### DESCRIPTION OF EXEMPLIFYING EMBODIMENTS

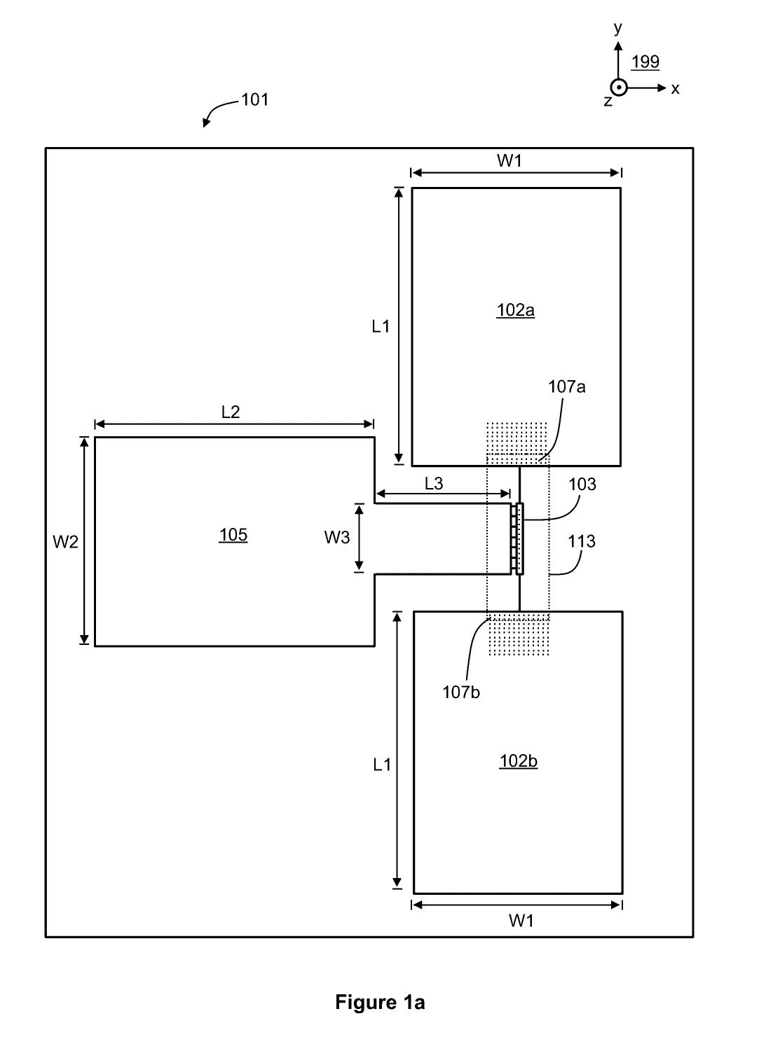

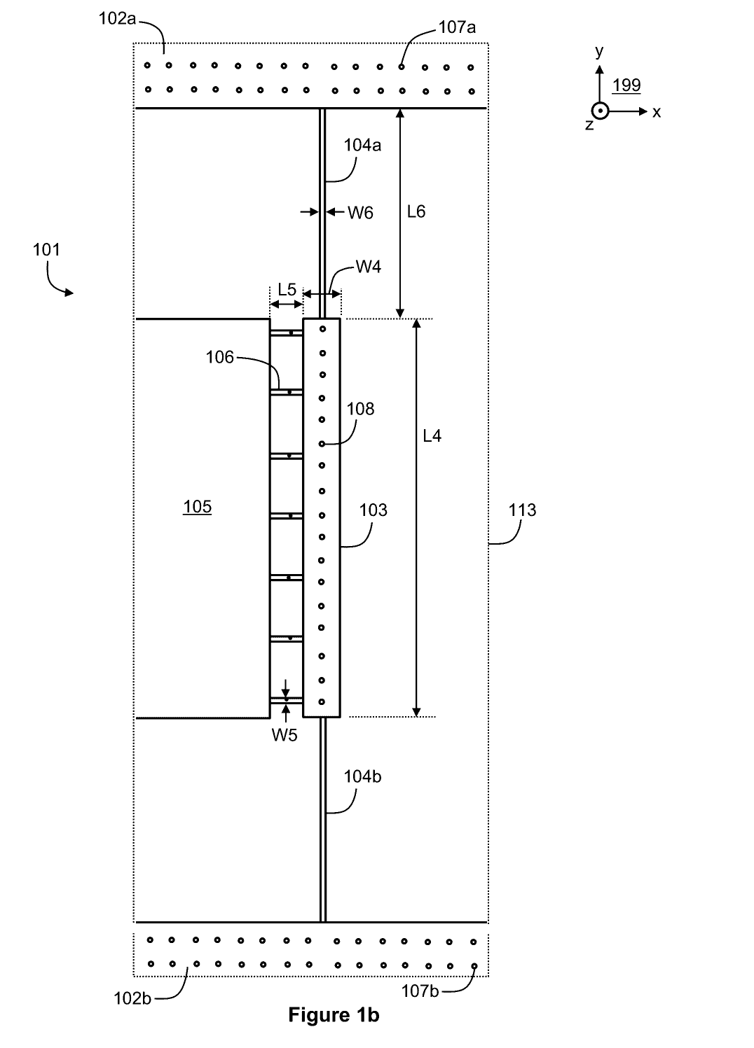

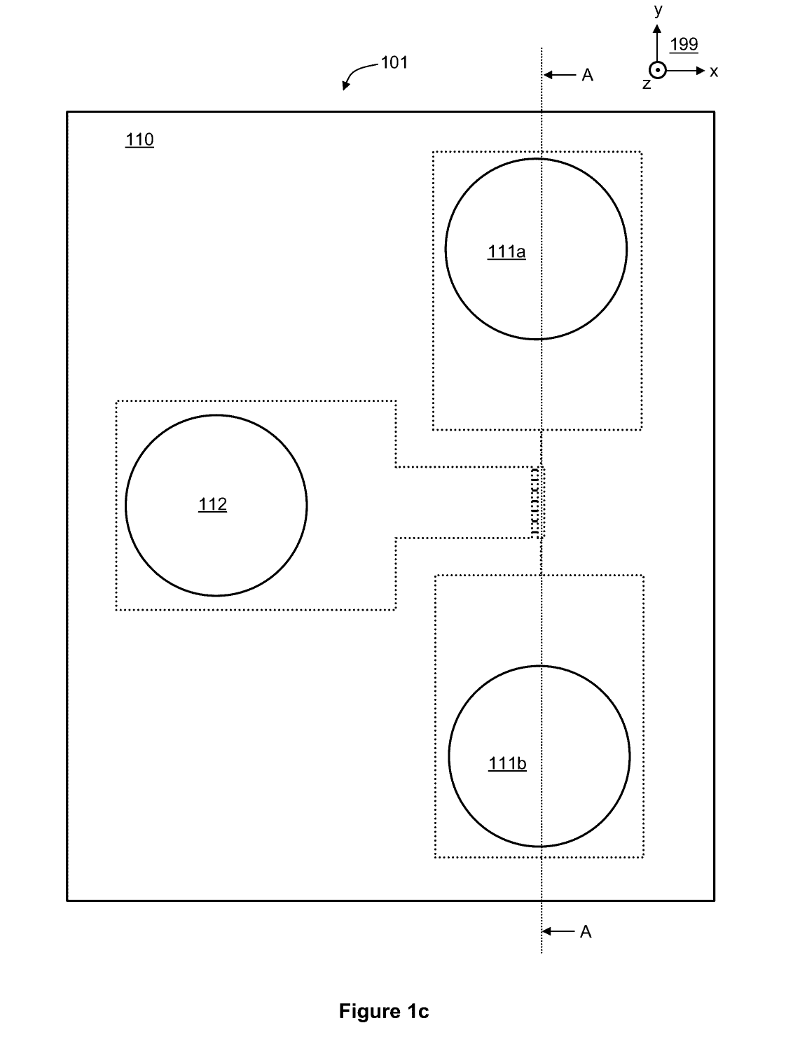

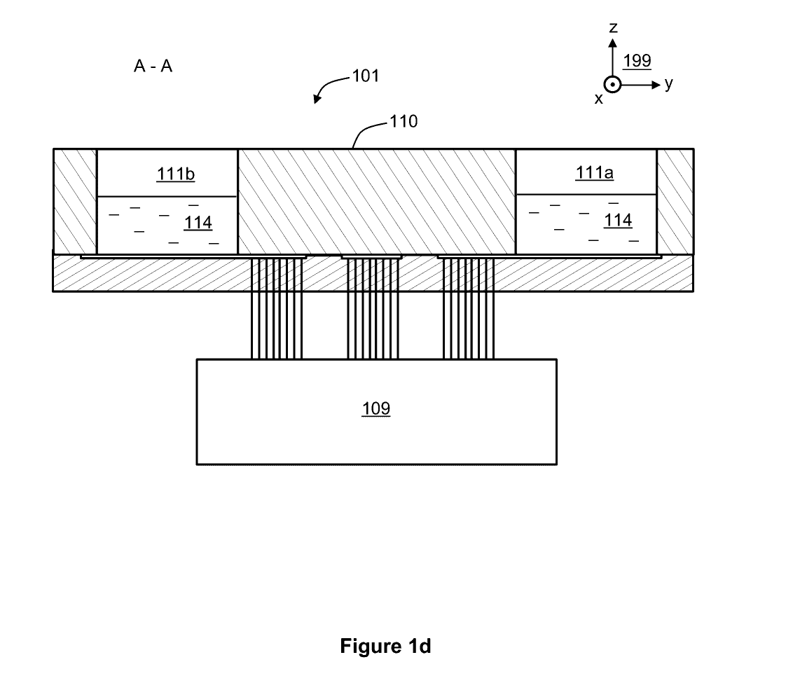

FIG. 1a shows a top-view of a cell culturing platform 101 according to an exemplifying and non-limiting embodiment of the invention, and FIG. 1b shows a magnification of a part 113 of FIG. 1a. The cell culturing platform 101 comprises solid material that is adapted to constitute first cell chambers 102a and 102b for containing somas of neuronal cells, a process chamber 103 connected to the first cell chambers with first channels, and a second cell chamber 105 for containing somas of myelinating cells and connected with second channels to the process chamber 103. In FIG. 1b, the first channels are denoted with reference numbers 104a and 104b, and one of the second channels is denoted with a reference number 106. The first channels 104a and 104b are suitable for guiding growth of the neuronal cell processes of the neuronal cells, i.e. the axons and dendrites of the neuronal cells, from the first cell chambers 102a and 102b to the process chamber 103 and for inhibiting the somas of the neuronal cells from moving from the first cell chambers to the process chamber. The second channels are suitable for guiding growth of the myelinating cell processes of the myelinating cells, i.e. the appendages of myelinating cells, from the second cell chamber 105 to the process chamber 103 and for inhibiting the somas of the myelinating cells from moving from the second cell chamber to the process chamber. The process chamber 103 constitutes a room for myelination of the neuronal cell processes by the myelinating cell processes of the myelinating cells. As the first and second channels are dimensioned to inhibit the cell somas from entering the process chamber 103 where the myelination takes place, the process chamber can be kept substantially free from the cell somas and thus the detection of the myelination is facilitated.

In a cell culturing platform according to an exemplifying and non-limiting embodiment of the invention, the sum of cross-sectional areas of the second channels is at least two, or preferably at least three, times greater than the sum of cross-sectional areas of the first channels. The cross-sections of the first channels are taken along a plane parallel to the xz-plane of a coordinate system 199, and the cross-sections of the second channels are taken along a plane parallel to the yz-plane of the coordinate system 199. The cross-sections of the first channels may have a rectangular form, or some other suitable form such as e.g. a half circle. Correspondingly, the cross-sections of the second channels may have a rectangular form, or some other suitable form such as e.g. a half circle. The average number of myelinating cell processes per each neuronal cell process is at least in some extent dependent on the ratio of the sum of the cross-sectional areas of the second channels and the sum of the cross-sectional areas of the first channels. There can be for example three or more second channels for each of the first channels, i.e. the number of the second channels can be three or more times greater than the number of the first channels.

In the exemplifying cell culturing platform illustrated in FIGS. 1a 1b, the process chamber 103 has an elongated shape and the first channels 104a and 104b are connected to the mutually opposite shorter sides of process chamber and all of the second channels are connected to one of the longer sides of the process chamber. The longer sides of the process channel can be for example at least three times longer than the shorter sides of the process channel. This design is advantageous for providing an efficient myelination process.

In a cell culturing platform according to an exemplifying and non-limiting embodiment of the invention, each of the first channels 104a and 104b is at least 50% longer than each of the second channels. The first channels should have a certain minimum length for being able to guide the growth of the neuronal cell processes towards the process chamber 103.

The dimensions of the first cell chambers 102a and 102b shown in FIG. 1a can be for example:
the width W1 on the range from 0.5 mm to 5 mm,
the length L1 on the range from 0.5 mm to 5 mm, and
the heights on the range from 50 μm to 10 mm, where the heights are substantially vertical when the cell culturing platform is in its operating position, i.e. the heights are parallel with the z-direction of the coordinate system 199.

The dimensions of the process chamber 103 shown in FIG. 1a can be for example:
the width W4 on the range from 50 μm to 150 μm,
the length L4 on the range from 0.5 mm to 3 mm, and
the height on the range from 50 μm to 150 μm, where the height is substantially vertical when the cell culturing platform is in its operating position, i.e. parallel with the z-direction of the coordinate system 199.

The dimensions of the second cell chamber 105 shown in FIG. 1a can be for example:
the width W2 on the range from 0.5 mm to 4 mm,
the length L2 on the range from 0.5 mm to 5 mm,
the width W3 on the range from 0.5 mm to 1.5 mm,
the length L3 on the range from 0 mm to 3 mm, and
the height on the range from 50 μm to 10 mm, where the height is substantially vertical when the cell culturing platform is in its operating position, i.e. parallel with the z-direction of the coordinate system 199.

The dimensions of the first channels 104a and 104b shown in FIG. 1b can be for example:
the width W6 on the range from 0.5 μm to 15 μm, preferably from 5 μm to 15 μm, and yet more preferably W6 is about 10 μm,
the length L6 on the range from 50 μm to 1000 μm, and
the height on the range from 0.5 μm to 5 μm, preferably 0.5 μm to 1.5 μm, and yet more preferably the height is about 1 μm, where the height is substantially vertical when the cell culturing platform is in its operating position, i.e. parallel with the z-direction of the coordinate system 199.

The dimensions of the second channel 106 and of the other second channels shown in FIG. 1b can be for example:
the width W5 on the range from 0.5 μm to 15 μm, preferably from 5 μm to 15 μm, and yet more preferably W5 is about 10 μm,
the length L5 on a range from 1 μm to 200 μm, and
the height on the range from 0.5 μm to 5 μm, preferably 0.5 μm to 1.5 μm, and yet more preferably the height is about 1 μm, where the height is substantially vertical when the cell culturing platform is in its operating position, i.e. parallel with the z-direction of the coordinate system 199.

A cell culturing platform according to an exemplifying and non-limiting embodiment of the invention is made of transparent material so as to enable optical inspection of the myelination of the neuronal cell processes. Thus, myelination in the process chamber can be detected using optical microscopy techniques. The transparent material can be for example polystyrene or polyvinyl chloride with or without copolymers, polyethylenes, polystyrene-acrylonitrile, polypropylene, polyvinylidine chloride, silicone eleastomers, or similar materials.

FIG. 1c shows a top view of the cell culturing platform when provided with a cover portion 110, and FIG. 1d shows a view of a section taken along the line A-A shown in FIG. 1c. The cover portion 110 adapted to constitute reservoirs 111a, 111b, and 112 for containing liquid-form cell culturing medium 114 and connected to the first cell chambers and to the second cell chamber as illustrated in FIGS. 1c and 1d. The purpose of the reservoirs is to contain such an amount of the cell culturing medium 114 that the first and second cell chambers 102a, 102b and 105 are prevented from getting dry for a sufficiently long time.

A cell culturing platform according to an exemplifying and non-limiting embodiment of the invention comprises electrodes and wirings for transferring electrical signals from and to neuronal cells.

Advantageously, there are electrodes at least in the first cell chambers 102a and 102b. In FIGS. 1a and 1b, two of electrodes are denoted with reference numbers 107a and 107b. Furthermore, there can be electrodes in the process chamber 103. One of the electrodes of the process chamber is denoted with a reference number 108 in FIG. 1b. Yet furthermore, there can be electrodes in the second channels between the second cell chamber 105 and the process chamber 103.

A cell culturing platform according to an exemplifying and non-limiting embodiment of the invention comprises a circuitry connected to the wirings and adapted to measure time elapsed between a first moment when an electrical signal appears on a first one of the electrodes and a second moment when a corresponding electrical signal appears on a second one of the electrodes. The above-mentioned circuitry is denoted with a reference number 109 in FIG. 1d. The complete myelination makes a signal in a neuronal cell about 15 times faster in vivo. Therefore, the myelination can be detected by measuring signal propagations times with the aid of the electrodes and the circuitry 109.

In the exemplifying cell culturing platform illustrated in FIGS. 1a-1d, there are the two first cell chambers 102a and 102b for the somas of the neuronal cells. The two first cell chambers are connected to mutually opposite ends of the process chamber 103. It is, however, also possible that there is only one cell chamber for the somas of the neuronal cells. On the other hand, it is also possible that there are more than two cell chambers for the somas of the neuronal cells.

Furthermore, a cell culture platform according to an exemplifying and non-limiting embodiment of the invention includes drug/medium application inlets in the first cell chambers 102a and 102b, in the second cell chamber 105, and/or in the process chamber 103 that facilitate providing drug/medium changes only to desired/dedicated areas of the cell culture platform.

Cell culturing platforms of the kind described above can be fabricated by using a slightly modified, i.e. different masktype and slower but more accurate process, version of rapid prototyping method which is commonly used in fabrication of Polydimethylsiloxane “PDMS” structures. In this method, the PDMS structure is molded by using an SU-8 mold. SU-8 is a commonly used epoxy-based negative photoresist. It is a very viscous polymer that can be spun or spread over a thickness ranging from below 1 micrometer up to above 300 micrometers and still be processed with standard contact lithography. Thus, the SU-8 mold can be fabricated by using standard lithography methods.

The SU-8 mold is fabricated by spin-coating SU-8 photoresist on top of e.g. silicon wafer, the height of the layer can be controlled by changing the spinning speed or viscosity of used SU-8. SU-8 is then hard baked and exposed to UV-light through a lithography mask. During the exposure, the features in the mask are transferred to the SU-8. SU-8 is then baked again and developed. This process is repeated multiple times as each height in the mold requires its own SU-8 layer.

Once the SU-8 mold is completed, the PDMS is molded in it. The PDMS components are mixed together by using 1:10 curing agent—base polymer ratio and poured onto the mold. The PDMS is then exposed to vacuum in order to remove air bubbles. After the vacuum treatment, the PDMS is baked in e.g. 60 degrees for 10 hours. After the bake, the PDMS is cut out of the mold and the necessary inlets for fluids are punched into it by using punching tools. Before using the PDMS structures, they are exposed to oxygen plasma in order to make them hydrophilic.

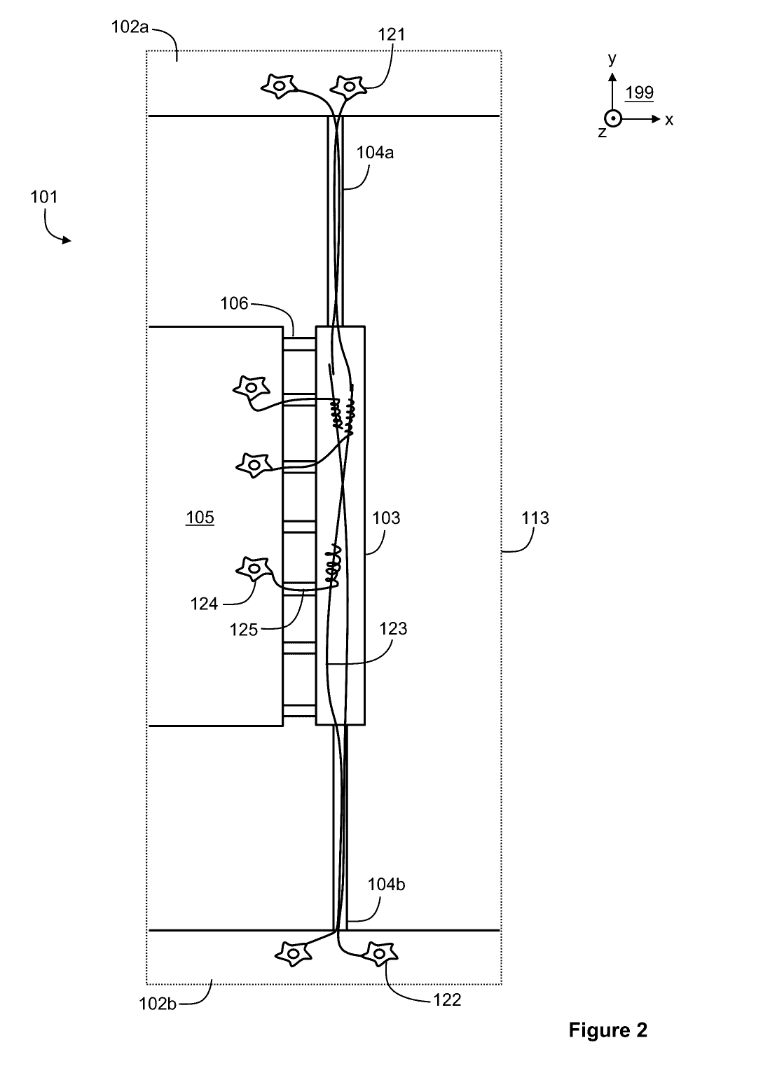

FIG. 2 illustrates a cell culture system according to an exemplifying and non-limiting embodiment of the invention for modeling the myelination in vitro. The cell culture system comprises a cell culturing platform 101 according to an embodiment of the invention. Each of the first cell chambers 102a and 102b contains somas of neuronal cells. In FIG. 2, two of the somas of the neuronal cells are denoted with reference numbers 121 and 122. The neuronal cell processes of the neuronal cells are capable of entering the process chamber 103 through the first channels 104a and 104b and of forming synapses with each other. One of the neuronal cell processes is denoted with a reference number 123. The second cell chamber 105 contains somas of myelinating cells. One of the somas of the myelinating cells is denoted with a reference number 124. Myelinating cell processes of the myelinating cells are capable of entering the process chamber 103 through the one or more second channels 106 and myelinating the neuronal cell processes of the neuronal cells in the process chamber. One of the myelinating cell processes is denoted with a reference number 125. The neuronal cells may comprise neurons and/or neural precursor cells of an animal or a human being, and the myelinating cells may comprise oligodendrocytes, oligodendrocyte precursor cells, and/or schwann cells of an animal or a human being.

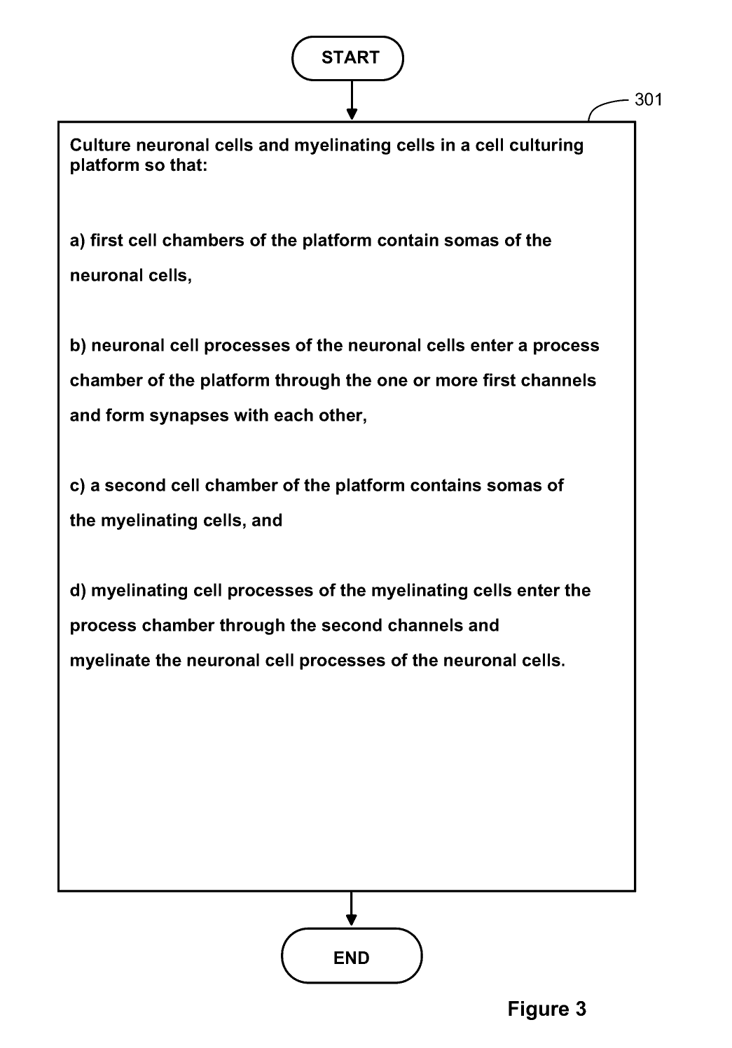

FIG. 3 shows a chart of a method according to an exemplifying and non-limiting embodiment of the invention for modeling the myelination in vitro. The method comprises culturing 301 neuronal cells and myelinating cells in a cell culturing platform according to an embodiment of the invention, wherein
each of the one or more first cell chambers contains somas of the neuronal cells,
neuronal cell processes of the neuronal cells enter the process chamber through the one or more first channels and form synapses with each other,
the second cell chamber contains somas of the myelinating cells, and
myelinating cell processes of the myelinating cells enter the process chamber through the one or more second channels and myelinate the neuronal cell processes of the neuronal cells in the process chamber.

In a method according to an exemplifying and non-limiting embodiment of the invention, the neuronal cells comprise neurons and/or neural precursor cells of an animal or a human being.

In a method according to an exemplifying and non-limiting embodiment of the invention, the myelinating cells comprise oligodendrocytes, oligodendrocyte precursor cells, and/or schwann cells of an animal or a human being.

In a method according to an exemplifying and non-limiting embodiment of the invention, the cell culturing platform is made of transparent material, and the method comprises optically inspecting, with the aid of a microscope, the process chamber so as to find out whether the myelinating cell processes of the myelinating cells have myelinated the neuronal cell processes of the neuronal cells.

In a method according to an exemplifying and non-limiting embodiment of the invention, the cell culturing platform comprises electrodes for directing electrical signals to the neuronal cells and for receiving electrical signals from the neuronal cells. The method according to this embodiment of the invention comprises measuring time elapsed between a first moment when an electrical signal appears on a first one of the electrodes and a second moment when a corresponding electrical signal appears on a second one of the electrodes, and comparing the measured time with a reference value so as to find out whether the myelinating cell processes of the myelinating cells have myelinated the neuronal cell processes of the neuronal cells.

### EXPERIMENTAL EXAMPLE CASES

1. Materials and Methods

1.1 Cells

Neurons and oligodendrocytes, so called myelinating cells, used in these experimental cases were differentiated from human embryonic stem cell line as described by Skottman 2010. Differentiation was performed according to methods previously published by Lappalainen et al. 2010, Sundberg et al. 2011. For oligodendrocyte differentiation, minor modifications were made to the published method. A list of the reference publications is presented at the end of this section “Experimental example cases”.

1.2 Polydimethylsiloxane “PDMS” Structures

PDMS structures were manufactured using soft lithography rapid prototyping techniques, which allow fast fabrication of prototype devices from PDMS and which are described by Wolfe and Whitesides 2005. The manufacturing process is simple; first a mold is made from SU-8 negative photoresist on top of a silicon wafer using standard photolithography techniques as described by Park et al. 2006. Once the mold is ready the PDMS, Sylgard 184, components are mixed together and poured into the mold. Vacuum is then used to degas the PDMS and once degassed the PDMS is baked in an oven. After baking, the PDMS is peeled off the mold and fluid inlets are punched into it using punching tools. Once the PDMS structure has inlets it is ready to be used.

1.3 Preparation of Microelectrode Arrays “MEA”

In-house MEAs were prepared as described by Ryynänen et al. 2011.

1.4 Preparation of Structures for Cell Culture

PDMS structures were either permanently or reversibly bonded onto glass coverslips or MEA using oxygen plasma treatment. Cell culture surface in the structure was coated with laminin for neurons and with mixture of laminin, collagen IV and nidogen for myelinating cells.

1.5 Cell Culture in Structures

A subset of neurospheres and spheres of myelinating cells at the differentiation age of 8-10 weeks were mechanically dissected into smaller aggregates and approximately 10 aggregates were plated on the cell chambers or on the second cell chamber. Neurons in the cell chambers were fed with medium described by Lappalainen et al. 2010 and myelinating cells in the second cell chamber with medium described by Sundberg et al 2011. Media were changed three times per week.

1.6 Analysis

i) Microscopy and Time-lapse Imaging

The growth and behavior of cells in the structure were investigated by phase contrast imaging and time-lapse imaging.

ii) Immunocytochemical Staining and Fluorescence Microscopy

The presence of neurons and myelinating cells in the culture was assessed by immunocytochemical staining. For this purpose, cells in the structure were fixed with 4% PFA and immunostained with markers specific for neurons and myelinating cells as described by Lappalainen et al, 2010 and Sundberg et al. 2011. Samples were viewed with fluorescence microscopy.

iii) MEA Signaling

Electrical activity of neurons in the structure was measured using a MEA system, i.e. Multi Channel Systems. Measurement and analysis was performed as described by Heikkilä et al. 2009.

2. Experimental Example Case 1

Somas of neurons are restricted to the cell chamber whereas neuronal cell processes are guided via first channels to the process chamber:

Neuron aggregates were plated onto the cell chambers of the structure and cultured until analyzed. Neurons were successfully cultured in the cell culturing platform. Somas of the neurons were restricted to the cell chamber and neuronal cell processes were able to enter the first channels and form network in the process chamber.

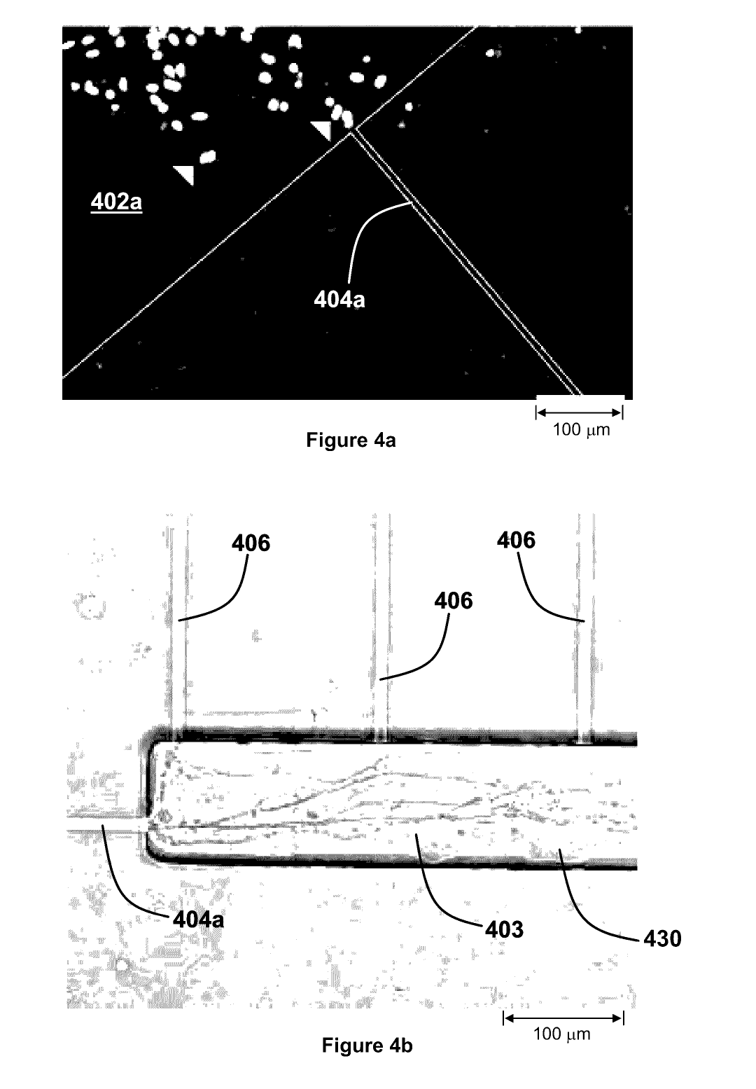

FIG. 4a shows the cell chamber 402a that contains the nuclei of the neurons some of which are denoted with white arrow heads. The nuclei are stained with 4′,6-diamidino-2-phenylindole “DAPI” nuclear stain. The somas do not enter the first channel 404a that is free of the somas.

FIG. 4b shows the first channel 404a on the left via which the neuronal cell processes enter the process chamber 403. The neuronal cell processes continue to grow along the process chamber. Small white arrows 430 point the growth direction. The second channels for entering of myelinating cell processes are denoted with a reference number 406.

3. Experimental Example Case 2

Restricted process growth of myelinating cells in the process chamber:

Myelinating cells were plated on the second cell chamber of the cell culturing platform and cultured until analyzed. Myelinating cells were successfully cultured in the second cell chamber and the processes of myelinating cells were able to enter process chamber via the second channels.

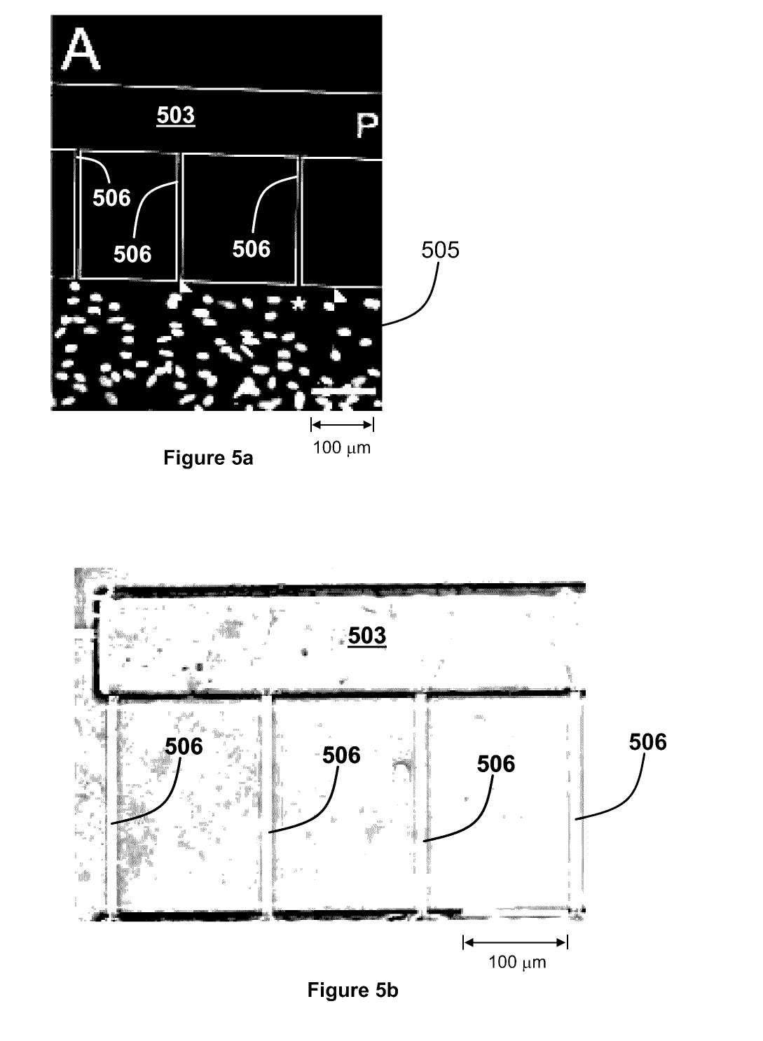

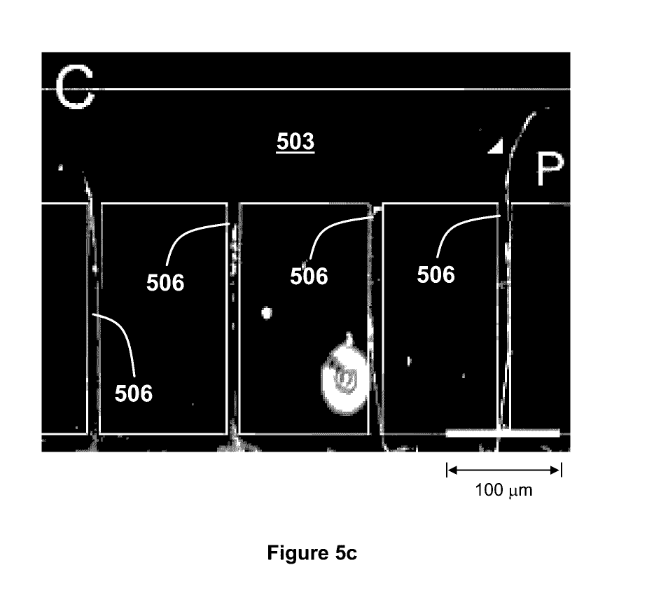

FIG. 5a shows the second cell chamber 505 that contains the nuclei of the myelinating cells some of which are denoted with white arrow heads. The nuclei are stained with 4′,6-diamidino-2-phenylindole “DAPI” nuclear stain. The somas do not enter the second channels 506 that are free of the somas. The process chamber is denoted with the reference number 503.

FIG. 5b shows the second channels 506 via which the myelinating cell processes enter the process chamber 503. Three of the myelinating cell processes are denoted with white arrow heads.

FIG. 5c shows immunocytochemical staining of the myelinating cell processes entering the process chamber 503 via the second channels 506. The staining is GalC staining. One of the myelinating cell processes is denoted with a white arrow head.

4. Experimental Example Case 3

Detection of neuronal activity from the process chamber:

Neuron aggregates were plated onto the cell chambers of the structure and cultured until analyzed. The cell culture platform was inserted on top of in house made MEA. The neuronal cell processes were able to enter the first channels and form network in the process chamber on top of MEA electrodes. The spontaneous activity of the formed neuronal network was measured with MEA.

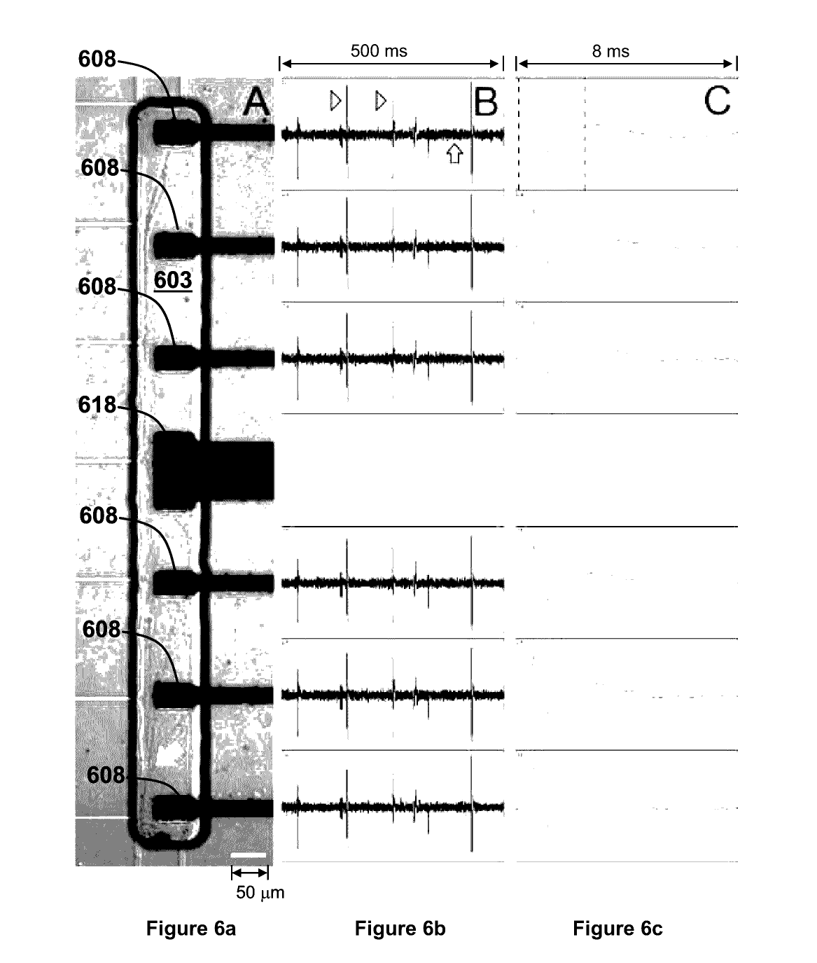

FIG. 6a shows the process chamber 603 containing 6 electrodes 608 and reference electrode 618. The neuronal cell processes grow along the process chamber. One of the neuronal cell processes is denoted with a white arrow.

FIG. 6b shows the action potentials measured from the individual electrodes in the process chamber. Two spikes representing the action potentials are denoted with white arrow heads and the background level is denoted with a white arrow. As can be seen, the action potentials are clearly distinguishable from the background level.

FIG. 6c shows a waveform with a typical action potential shape detected during the measurement illustrated with the aid of FIGS. 3a and 3b. The waveform of the action potential shape is between vertical dashed lines shown in FIG. 6c. The same action potential waveform was measured from all the electrodes in the process chamber 603, thus the individual signal was able to propagate through all electrodes in the process chamber.

5. List of the Reference Publications

Skottman H. 2009, Derivation and characterization of three new human embryonic stem cell lines in Finland, In Vitro Cell. Dev. Biol.—Animal, 46:206-209.

Heikkilä T, Ylä-Outinen L, Tanskanen J, Lappalainen R, Skottman H, Suuronen R, Mikkonen J, Hyttinen J, Narkilahti S. 2009, Human embryonic stem cell-derived neuronal cells form spontaneously active neuronal networks in vitro. Exp Neurol. 218 (1):109-16.

Lappalainen R., Salomäki M., Ylä-Outinen L., Heikkilä T J., Hyttinen J A K., Pihlajamäki H., Suuronen R., Skottman H., Narkilahti S. 2010 Similarly derived and cultured hESC lines show variation in their developmental potential towards neuronal cells in long-term culture. Regen. Med. 5:749-62.

Park, J. W., Vahidi, B., Taylor, A., Rhee, S. W., Jeon, N. L. Microfluidic culture platform for neuroscience research. Nature protocols, vol. 1, no. 4, 2006, pp. 2128-2136.

Ryynänen Tomi, Kujala Ville, Ylä-Outinen Laura, Korhonen Ismo, Tanskanen Jarno M A, Kauppinen Pasi, Aalto-Setälä Katriina, Hyttinen Jan, Kerkelä Erja, Narkilahti Susanna, Lekkala Jukka. 2011, All-Titanium Microelectrode Array for Field Potential Measurements from Neurons and Cardiomyocytes—a Feasibility Study. Micromachines 2, 394-409.

Sundberg M., Hyysalo A., Skottman H., Shin S., Vemuri M., Suuronen R., Narkilahti S. 2011, A xeno-free culturing protocol for pluripotent stem cell-derived oligodendrocyte precursor cell production. Regenerative Medicine. 6 (4):449-60.

Wolfe, D., Whitesides, G. Rapid prototyping of functional microfabricated devices by soft lithography. Nanolithography and Patterning Techniques in Microelectronics, Woodhead Publishing Limited, 2005, pp. 76-119.

The non-limiting, specific examples provided in the description given above should not be construed as limiting the scope and/or the applicability of the appended claims.

1-25. (canceled)

26. A cell culturing platform comprising solid material adapted to constitute:
one or more first cell chambers for containing somas of neuronal cells, and
a process chamber connected to the one or more first cell chambers with one or more first channels,
wherein:
the one or more first channels are suitable for guiding growth of neuronal cell processes of the neuronal cells from the one or more first cell chambers to the process chamber and for inhibiting the somas of the neuronal cells from moving from the one or more first cell chambers to the process chamber,
the process chamber constitutes a room for myelination of the neuronal cell processes by myelinating cell processes of myelinating cells,
the solid material is further adapted to constitute a second cell chamber for containing somas of the myelinating cells and connected with one or more second channels to the process chamber,
the second channels are suitable for guiding growth of the myelinating cell processes of the myelinating cells from the second cell chamber to the process chamber and for inhibiting the somas of the myelinating cells from moving from the second cell chamber to the process chamber, and
the process chamber has an elongated shape so that each of the one or more first channels is connected to one of shorter sides of process chamber and each of the one or more second channels is connected to one of longer sides of the process chamber, the longer sides of the process channel being at least three times longer than the shorter sides of the process channel.

27. A cell culturing platform according to claim 26, wherein a sum of cross-sectional areas of the one or more second channels is at least two times greater than a sum of cross-sectional areas of the one or more first channels.

28. A cell culturing platform according to claim 26, wherein there are at least two second channels for each of the one or more first channels.

29. A cell culturing platform according to claim 26, wherein each of the one or more first channels is at least 50% longer than each of the one or more second channels.

30. A cell culturing platform according to claim 26, wherein the solid material is adapted to constitute two first cell chambers and the two first cell chambers are connected to mutually opposite ends of the process chamber.

31. A cell culturing platform according to claim 26, wherein the cell culturing platform comprises electrodes and wirings for directing electrical signals to the neuronal cells and for receiving electrical signals from the neuronal cells.

32. A cell culturing platform according to claim 30, wherein the cell culturing platform comprises one or more electrodes located in one of the two first cell chambers, one or more electrodes located in another of the two first cell chambers, and wirings for directing electrical signals to the neuronal cells and for receiving electrical signals from the neuronal cells.

33. A cell culturing platform according to claim 31, wherein the cell culturing platform comprises a circuitry connected to the wirings and adapted to measure time elapsed between a first moment when an electrical signal appears on a first one of the electrodes and a second moment when a corresponding electrical signal appears on a second one of the electrodes.

34. A cell culturing platform according to claim 26, wherein the solid material is transparent so as to enable optical inspection of the myelination of the neuronal cell processes.

35. A cell culturing platform according to claim 26, wherein the cell culturing platform further comprises a cover portion adapted to constitute reservoirs for containing liquid-form cell culturing medium and connected to the one or more first cell chambers and to the second cell chamber.

36. A cell culture system for modeling myelination in vitro, the cell culture system comprising a cell culturing platform comprising solid material adapted to constitute:
one or more first cell chambers for containing somas of neuronal cells, and
a process chamber connected to the one or more first cell chambers with one or more first channels,
wherein:
the one or more first channels are suitable for guiding growth of neuronal cell processes of the neuronal cells from the one or more first cell chambers to the process chamber and for inhibiting the somas of the neuronal cells from moving from the one or more first cell chambers to the process chamber,
the process chamber constitutes a room for myelination of the neuronal cell processes by myelinating cell processes of myelinating cells,
the solid material is further adapted to constitute a second cell chamber for containing somas of the myelinating cells and connected with one or more second channels to the process chamber,
the second channels are suitable for guiding growth of the myelinating cell processes of the myelinating cells from the second cell chamber to the process chamber and for inhibiting the somas of the myelinating cells from moving from the second cell chamber to the process chamber,
the process chamber has an elongated shape so that each of the one or more first channels is connected to one of shorter sides of process chamber and each of the one or more second channels is connected to one of longer sides of the process chamber, the longer sides of the process channel being at least three times longer than the shorter sides of the process channel,
each of the one or more first cell chambers contains the somas of the neuronal cells,
the neuronal cell processes of the neuronal cells are capable of entering the process chamber through the one or more first channels and forming synapses with each other,
the second cell chamber contains the somas of the myelinating cells, and
the myelinating cell processes of the myelinating cells are capable of entering the process chamber through the one or more second channels and myelinating the neuronal cell processes of the neuronal cells in the process chamber.

37. A cell culture system according to claim 36, wherein the neuronal cells comprise neurons and/or neural precursor cells of one of the following: an animal, a human being.

38. A cell culture system according to claim 36, wherein the myelinating cells comprise at least one of the following: oligodendrocytes, oligodendrocyte precursor cells, schwann cells of an animal or human being.

39. A method for modeling myelination in vitro, the method comprising culturing neuronal cells and myelinating cells in a cell culturing platform comprising solid material adapted to constitute:
one or more first cell chambers for containing somas of the neuronal cells, and
a process chamber connected to the one or more first cell chambers with one or more first channels,
wherein:
the one or more first channels are suitable for guiding growth of neuronal cell processes of the neuronal cells from the one or more first cell chambers to the process chamber and for inhibiting the somas of the neuronal cells from moving from the one or more first cell chambers to the process chamber,
the process chamber constitutes a room for myelination of the neuronal cell processes by myelinating cell processes of the myelinating cells,
the solid material is further adapted to constitute a second cell chamber for containing somas of the myelinating cells and connected with one or more second channels to the process chamber,
the second channels are suitable for guiding growth of the myelinating cell processes of the myelinating cells from the second cell chamber to the process chamber and for inhibiting the somas of the myelinating cells from moving from the second cell chamber to the process chamber,
the process chamber has an elongated shape so that each of the one or more first channels is connected to one of shorter sides of process chamber and each of the one or more second channels is connected to one of longer sides of the process chamber, the longer sides of the process channel being at least three times longer than the shorter sides of the process channel,
each of the one or more first cell chambers contains the somas of the neuronal cells,
the neuronal cell processes of the neuronal cells enter the process chamber through the one or more first channels and form synapses with each other,
the second cell chamber contains the somas of the myelinating cells, and
the myelinating cell processes of the myelinating cells enter the process chamber through the one or more second channels and myelinate the neuronal cell processes of the neuronal cells in the process chamber.

40. A method according to claim 39, wherein the neuronal cells comprise neurons and/or neural precursor cells of one of the following: an animal, a human being.

41. A method according to claim 39, wherein the myelinating cells comprise at least one of the following: oligodendrocytes, oligodendrocyte precursor cells, schwann cells of an animal or human being.

42. A method according to claim 39, wherein the solid material is transparent so as to enable optical inspection of the myelination of the neuronal cell processes, and the method comprises optically inspecting, with the aid of a microscope, the process chamber so as to find out whether the myelinating cell processes of the myelinating cells have myelinated the neuronal cell processes of the neuronal cells.

43. A method according to claim 39, wherein the cell culturing platform comprises electrodes and wirings for directing electrical signals to the neuronal cells and for receiving electrical signals from the neuronal cells, and the method comprises measuring time elapsed between a first moment when an electrical signal appears on a first one of the electrodes and a second moment when a corresponding electrical signal appears on a second one of the electrodes, and comparing the measured time with a reference value so as to find out whether the myelinating cell processes of the myelinating cells have myelinated the neuronal cell processes of the neuronal cells.
